(19) United States
(12) Patent Application Publication
(10) Pub. No.: US 2005/0276826 A1
(43) Pub. Date: Dec. 15, 2005

(54) Candle for delivery of massage oil

(76) Inventors: Robert A. Culver, Seattle, WA (US); Tina L. Benshoof, Shoreline, WA (US); Matthew A. Applebach, Seattle, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 10/922,615

(22) Filed: Aug. 19, 2004

Related U.S. Application Data
(60) Provisional application No. 60/496,559, filed on Aug. 19, 2003.

(51) Int. Cl. A61K 7/00

(52) U.S. Cl. 424/401

(57) ABSTRACT

A delivery method and apparatus for massage oil is provided along with an associated massage oil composition. The massage oil is provided in a solid state at room temperature. The massage oil is heated to a liquid state at a temperature which is skin safe. The massage oil can then be applied to the skin and massaged into the skin. The massage oil can be provided in candle form to facilitate melting of the massage oil from the solid state to the liquid state. Compositions including primarily an oil such as hydrogenated soybean oil, palm kernel oil, coconut oil or cocoa butter, which are solid at room temperature, provide a major constituent for the massage oil composition disclosed. Fragrances, emollients, moisturizers, sweeteners, flavorants or other constituents can also be included.

### CROSS-REFERENCE TO RELATED APPLICATIONS

This application claims benefit under Title 35, United States Code §119(e) of U.S. Provisional Application No. 60/496,559 filed on Aug. 19, 2003.

### FIELD OF THE INVENTION

The following invention relates to preparations such as massage oils which can be rubbed into the skin. More particularly, this invention relates to preparations and methods for their application which are solid at room temperature but which can be melted at a temperature which is skin safe and with the melted preparation suitable for being massaged into the skin.

### BACKGROUND OF THE INVENTION

Massage oils have long been known in the art for enhancing the desirability of a massage and to beneficially condition the skin of the subject individual. In particular, massage oils themselves can beneficially alter friction between the massager's hands and the skin being massaged and enhance the tactile sensory perception of the individual receiving the massage. In addition, the massage oil can contain a fragrance which is desirable and/or therapeutic. Hence, during the massage the aroma associated with the fragrance enhances the overall experience for the individual receiving the massage.

Such massage oils (also called body oils) and lotions are liquid at room temperature and contain molecular structures which are susceptible to attack and degradation by atmospheric oxygen and other free radicals, leading to the oils becoming rancid and spoilage of the product. Lotions, due to their high water content (typically 80-90%), require that anti-microbial compounds be added to retard the growth of microbes that could cause product spoilage or compromise product safety.

Such oils and lotions generally have physical properties that allow them to be easily applied to the skin, being liquids or heavy emulsions. Because liquids are difficult to contain, sometimes these liquids escape their containers and can cause unintended damage.

Body oils and lotions are often heated to body temperature before being applied by a massage therapist. Therefore, it would be advantageous for a product to have qualities that resist spoilage and spillage as well as enhancing the sensory and therapeutic values associated with massage, massage therapy, aroma therapy and skin conditioning.

Candles have long been known in the art, particularly as a method for providing lighting. Candles also can provide a source of heat and can be made of a wax or related material which contains a desirable aroma which is released to a greater extent as the wax or other material is melted by the flame on the wick of the candle.

Some specialty candles are known in the art for utilization to provide some form of therapeutic benefit. For instance, U.S. Pat. No. 1,264,781 to Ehrhardt teaches a means for the application of solid pharmaceutical preparations or products. With this invention the pharmaceutical preparation is provided in a solid form and a wick is provided. When the wick is ignited the solid material is melted and then can be applied directly to the skin.

In examples taught by Ehrhardt, the material being applied is identified by the name “Ambrine.” A method taught by Ehrhardt involves dripping the Ambrine that is melted by the flame on the wick of the candle, onto a wound. Such wounds are taught to include burns, surgical sites or for treatment of other afflictions where treatment by heat is desired.

Important to note in the method taught by Ehrhardt is that the Ambrine or other material is taught as hardening upon cooling to body temperature to produce an adherent covering layer which is somewhat flexible and remains as a covering upon the skin. Furthermore, the Ambrine is disclosed as being primarily a paraffin substance, rather than an oil substance which is capable of being rubbed into or absorbed into skin. Nothing in Ehrhardt teaches or fairly suggests any rubbing or massaging of the material applied to the skin in the method taught by Ehrhardt. Thus, candles have not been known in the art as a particular method for delivering massage oil directly to the skin of an individual for use as a massage oil thereon.

### SUMMARY OF THE INVENTION

With this invention, a massage oil preparation is provided which is easy to handle before use, resists spoilage, and which is easy to apply in an enjoyable fashion for use on the skin of an individual. In particular, a massage oil preparation is disclosed which is a composition which is a majority hydrogenated soy oil or some other oil, such as coconut oil or palm kernel oil, which has a suitable melting point. This melting point should be such that the oil is hardened into a solid somewhat waxy form at room temperature and remains solid at temperatures encountered in typical storage and transport environments. At some higher temperature which is still safe for direct application to the skin, the oil melts into a liquid state.

The preparation preferably includes additional substances which are particularly released when the oil melts. These additional constituents preferably include a fragrance and/or an emollient, and/or a moisturizer, and optionally a flavorant. The preparation is preferably provided within a container from which the preparation can be poured and in which the preparation can reharden when restorage for later use is desired. In at least one form of the invention, a wick is provided which can be ignited to provide the heat necessary to melt the oil and associated constituents within the preparation.

When the oil has been melted, it can be poured from the container in appropriate amounts directly onto skin to be massaged. Alternatively, the melted oil can be poured onto the hands of the massager and then applied to the skin of the individual receiving the massage. The oil is then beneficially rubbed into the skin, typically by hand massage action, with the massaging action further releasing the fragrance and working any emollient and/or moisturizer into the skin, while also beneficially enhancing the quality of the massage.

### OBJECTS OF THE INVENTION

Accordingly, a primary object of the present invention is to provide a massage oil which is solid at room temperature and liquid at an elevated skin safe temperature.

Another object of the present invention is to provide a massage oil which avoids spoilage or other degradation during storage.

Another object of the present invention is to provide a massage oil which can be easily contained during storage and transport in solid form before being melted for use.

Another object of the present invention is to provide a massage oil which can be melted with an ignited wick in the form of a candle.

Another object of the present invention is to provide a massage oil preparation which is easy and safe to manufacture, store and use.

Another object of the present invention is to provide a method for delivering a massage oil which is exotic and stimulating for the participants.

Another object of the present invention is to provide a method for applying a skin preparation which involves skin safe temperatures.

Another object of the present invention is to provide a method for applying a skin preparation which easily allows for storage of unused portions for later use.

Other further objects of the present invention will become apparent from a careful reading of the included drawing figures, the claims and detailed description of the invention.

### BRIEF DESCRIPTION OF THE DRAWINGS

FIG. 1 is a perspective view of the candle of this invention showing the massage oil in solid form surrounding a wick within a container for the candle.

FIG. 2 is a full sectional view taken along line 2-2 of FIG. 1 and revealing interior details of the candle of this invention.

FIG. 3 is a perspective view of an alternative embodiment of that which is shown in FIG. 1 where no wick is provided and the massage oil is melted within the container by providing heat from an external source.

FIG. 4 is a full sectional view taken along line 4-4 of FIG. 3 and revealing interior details of the alternative embodiment shown in FIG. 3.

### DESCRIPTION OF THE PREFERRED EMBODIMENT

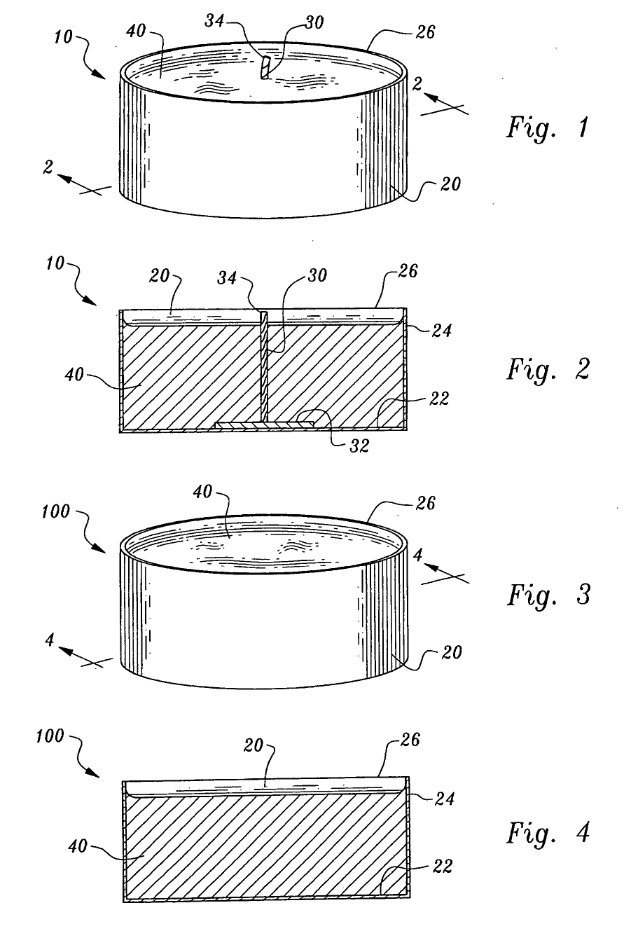

Referring to the drawings, wherein like reference numerals represent like parts throughout the various drawing figures, reference numeral 10 is directed to a candle (FIGS. 1 and 2) providing a preferred form of a configuration for delivery of massage oil 40 to skin of an individual according to the preferred method of this invention. The massage oil 40 is initially provided in a solid state and is then melted into a liquid state before being applied to the skin and massaged into the skin. The candle 10 thus provides the dual function of storing the massage oil 40 before use and delivering the massage oil in liquid form when ready for use. The candle 10 simultaneously creates a desirable atmosphere through the light provided by the ignited candle, as well as fragrances and other constituents optionally provided within the candle 10.

In essence, the composition of the massage oil 40 is primarily an oil which is in a solid state at room temperature and is in a liquid state at an elevated but safe temperature for application to skin. The massage oil 40 composition preferably additionally includes fragrances, emollients, and other constituents to provide the particular attributes for the massage oil 40 when massaged into the skin, as well as desirable fragrances, colors (and optionally flavors) to maximize the quality and nature of the sensational experience provided by the candle 10.

The candle 10 is preferably provided within a container 20 with a wick 30 optionally provided for melting of the massage oil 40 from a solid state to a liquid state. The container 20 conveniently can be tipped to allow the massage oil 40 in its liquid state to be applied, such as by pouring, onto the skin. Finally, the massage oil 40 is rubbed into the skin in a fashion analogous to use of known body oils using known massage procedures.

More specifically, and with particular reference to FIGS. 1 and 2, particular details of the structural configuration of the candle 10 of the preferred embodiment are described. While the candle 10 could be provided without the container 20, the container 20 is beneficially provided to house the massage oil 40 both in the solid state and in the liquid state. The container 20 can be any desired shape, with a generally cylindrical shape shown in FIGS. 1 and 2. The container 20 shown includes a generally flat circular floor 22. A side wall 24 extends cylindrically up from the floor 22 to a rim 26. A lid can optionally be provided to cover the container 20. When such a lid is provided, with the container 20 as shown in FIGS. 1 and 2, the lid would preferably be cylindrical with a circular lip extending down outboard of the rim 26 of the container 20 and with the cover either having a flat surface or a domed surface which would slide onto the container 20 adjacent the rim 26.

However, any container shape can be provided for housing of the massage oil 40 forming the candle 10. If no container 20 is provided, preferably a diameter of the candle 10 is sufficient so that a perimeter edge of the massage oil 40 remains in a solid state to contain massage oil 40 in a liquid state closer to a center of the candle 10.

The wick 30 provides a preferred form of a means to heat the massage oil 40 and transition the massage oil 40 from a solid state to a liquid state. The wick 30 preferably extends vertically from a holder 32 adjacent the floor 22 of the container 20 and up through a center of the container 20 to a tip 34. The tip 34 preferably is spaced from the holder 32 a distance similar to a distance from the floor 22 of the container 20 to the rim 26 of the container 20. The wick 30 can thus be ignited and the flame on the wick 30 will burn down to the upper surface of the massage oil 40 of the candle 10. This massage oil 40 then rapidly melts into a liquid state forming a pool of massage oil 40 available to be poured out of the container 20, or otherwise away from the candle 10 and onto the skin of an individual subject.

While the wick 30 is shown as a preferred form of means to heat the massage oil 40, other melting techniques could be utilized. For instance, and as particularly shown in FIGS. 3 and 4, a wickless candle 100 of massage oil 40 can be provided similar to the candle 10 of the preferred embodiment, but without the wick 30. A heat source such as an infrared lamp or other form of radiant heat source can be provided adjacent the candle 100. As the heat is applied, the massage oil 40 melts adjacent an upper portion thereof. The massage oil 40 can then be utilized, such as by pouring onto skin to be massaged.

Other heat sources could include conduction heat sources such as a water bath with heat radiating through the container 20. In such conduction heating embodiments, it is desirable that the container 20 be formed of metal or other material which facilitates heat transfer therethrough. In the final embodiment, the massage oil can be provided in small pebbles, referred to as pastilles. The pastilles can be placed into a melter as is commonly used in the aroma therapy arts, and the massage oil can be melted from the hardened state into a liquid state with only the amount of massage oil melted that is expected to be needed. In such an embodiment, the massage oil would originally be provided in solid chunks, such as pebbles, cubes or the like, which could be contained within various different containers suitable for holding the pastilles having such size and shape.

The use and operation of the candle 10, wickless candle 100 or other embodiments of the massage oil delivery system of this invention are described as follows. Initially, the massage oil 40 is provided in solid form. When in this solid form, the massage oil is easily stored and handled without loss of the massage oil 40 or causing spillage or other mess or damage to surrounding areas. Furthermore, in a solid state the massage oil 40 is less likely to become rancid or be infiltrated with bacteria, or experience other harmful contamination, separation or deterioration. The massage oil 40 in its solid state can thus be stored more easily and for a longer period without degradation of performance than would be the case with massage oil provided in liquid form.

When the massage oil 40 is ready to be used, the massage oil 40 is heated up to its melting point. As discussed above, such heating can occur through igniting the wick 30 of the candle 10 of FIGS. 1 and 2. Other heating methods described above could also be utilized, until at least a portion of the massage oil 40 has melted into a liquid state. Preferably, a melting temperature for the massage oil 40 is provided which is high enough to avoid inadvertent melting of the massage oil 40 during handling or storage and low enough so that the liquid massage oil can be applied to the skin without burning the skin or being at an unpleasantly hot temperature. This particular melting point can change based on the type of massage oil 40 provided and the particular constituents included within the massage oil 40. Also, atmospheric conditions, such as atmospheric pressure may have a slight impact on the melting point of the massage oil 40. Different individual users may have different tolerances to different heat levels altering the acceptable range of melting point for the massage oil 40. In general, a range of between 80° F. and 130° F. is considered a general range in which the melting point should be provided. Most preferably this range would be between 110° F. and 125° F., with the massage oil 40 in at least one example described below being provided at about 119° F., and providing effective results.

Once an appropriate amount of the massage oil 40 has been melted, it is desirable to avoid heating the massage oil 40 too much above its melting point temperature. Hence, it may be desirable to blow out the flame on the wick 30, or otherwise remove the massage oil 40 from away from the heat source which has melted the massage oil 40.

The massage oil 40 is then applied to the skin to be massaged. Most preferably, such application is provided by pouring the massage oil 40 out of the container 20 of the candle 10 of the preferred embodiment or the wickless candle 100 (FIGS. 3 and 4). Such pouring can be in drops or in a steady stream. The massage oil 40 could alternatively be poured onto hands to be used in the massage or applied to other massage apparatus, or otherwise transferred to the skin to be massaged through intermediate structures. The massage oil 40 is finally massaged into the skin, such as with a rubbing action or with other action involving movement of hands or other massage implements over the surfaces of the skin.

Various different constituents within the massage oil 40 can provide various different benefits to the user. The primary constituent of the massage oil 40 preferably provides the functional benefits associated with use of massage oil in general. Specifically, the massage oil can alter and improve the tactile sensation of nerves within the skin being massaged, and can alter a coefficient of friction involved in the massage in a desirable fashion, as well as soften and moisturize the skin.

Constituents within the massage oil 40 such as emollients, further can penetrate the skin to soften and soothe the skin. Other constituents that can be added include moisturizers which counteract any undesirable dryness in the skin. Other constituents could similarly be provided in the massage oil which beneficially treat undesirable skin conditions by being directly applied to the skin.

In addition, constituents can be added to otherwise enhance the sensational experience associated with the massage. For instance, fragrances can be added to the massage oil. These fragrances would be somewhat present when the massage oil is in the hardened state, and then be released into the air surrounding the massage oil to a greater extent when the massage oil is heated into a liquid state. Finally, massaging the massage oil into the skin would further cause release of the desirable aromas associated with the fragrance agents added to the massage oil.

Also, flavorants can be added to the massage oil composition to further add the sensation of taste to the overall massage experience. The flavorant would preferably be suitable for human consumption and typically would be a sweetener, but could provide other taste sensations (i.e. tart, salty, etc.). A preferred sweetener to utilize is Neotame. Alternative sweeteners include Alitame and Aspartame. Due to the relative sweetness of the sweeteners, substituting Neotame with Alitame would require about ten times more Alitame than Neotame, and substituting Neotame with Aspartame would require about one hundred times more Aspartame than Neotame. The flavor selected can match the fragrance if desired (i.e. a chocolate fragrance and a chocolate flavorant used together). In this way, many different senses can be desirably stimulated to maximize the overall quality of the massage experience.

The particular compositions making up the massage oil 40 of the candle 10 of the preferred embodiment and the wickless candle 100 of FIGS. 3 and 4, are described as various alternative embodiments to provide non-limiting examples of compositions which can effectively be provided according to this invention. In general, the compositions include a majority of an oil which is in a solid state at room temperature and which can be melted into a liquid state at an elevated but skin safe temperature. This majority constituent oil, or combination of oils, can include as examples one or more of hydrogenated soybean oil, palm kernel oil, or coconut oil. Hydrogenated soybean oil is also commonly referred to as soy wax.

The oil constituent can be at least partially a paraffin or other wax when in its solid state, or can be referred to as a “wax” while in fact not including any constituents that would technically be considered to be paraffins. Various other oils could also be utilized, provided that they have the appropriate melting point, discussed in detail above.

Most preferably, the oil providing the primary constituent for the overall massage oil composition provides ninety percent or more of the overall massage oil composition. In the embodiments and examples described in detail below, the remainder of the various massage oil compositions are described in detail. These examples are provided as non-limiting examples of compositions which have been shown to be effective in the practice of this invention.

### EXAMPLE 1

One hundred eighty-five (185) grams of soy wax were heated until molten, approximately 55° C. (131° F.). Seven (7) grams of essential oil of lavandula hybrida (hybrid lavender) was added to the soy wax as a fragrance while the soy wax was still in its molten state, and thoroughly incorporated with the soy wax.

A small amount of the soy wax/fragrance mixture was poured into the bottom of a glass container. While the soy wax/fragrance mixture was still partially molten a wick with a standard retainer base was inserted and centered in the container. The soy wax/fragrance was allowed to harden further and the remaining molten soy wax/fragrance mixture was added to the container. Once the soy wax/fragrance mixture had fully hardened the wick was trimmed. See FIGS. 1 and 2 showing a solid massage oil with integrated heating device.

The massage oil could be easily transported without any fear of spillage.

To use this massage oil the wick was ignited. After a period of time a small amount of the solid oil became molten and the temperature was appropriate for using small amounts for massage, massage therapy, aroma therapy and skin conditioning.

To transport this oil, the flame was extinguished and the oil was allowed to return to its non-molten state. The fragrance remained entrained within the hardened soy wax.

### EXAMPLE 2

Ninety-two (92) grams of coconut oil were heated until molten, approximately 30° C. (86° F.). Fourteen (14) grams of oil from oenothera biennis (evening primrose) were added as an emollient. Two (2) grams of essential oil from mentha piperita (peppermint) and two (2) grams of essential oil from anthemis nobilis (roman chamomile) were combined and added as a fragrance to the coconut oil while the coconut oil was in its molten state, and thoroughly incorporated with the coconut oil.

The coconut oil/emollient/fragrance mixture was poured into a metal container with a slip lid. The coconut oil/emollient/fragrance mixture was allowed to fully harden and the lid was placed on the container. The massage oil could be easily transported without fear of spillage. See FIGS. 3 and 4 showing a solid massage oil for use with external heating device. The emollient is very high in vitamin E which would tend to retard any tendency for this product to experience spoilage.

To use this massage oil, the lid was removed from the container and the container was placed under an infrared heating source such as a heat lamp. After a short period of time, a small amount of the solid oil became molten and the temperature was appropriate for using small amounts for massage, massage therapy, aroma therapy and skin conditioning.

To transport this oil, the container was removed from under the heat source and the massage oil allowed to return to its non-molten state.

### EXAMPLE 3

One hundred forty-two (142) grams of palm kernel oil were heated until molten, approximately 45° C. (131° F.). Twenty-eight (28) grams of oil from oenothera biennis (evening primrose) were added as an emollient. Nineteen (19) grams of essential oil from santalum album (sandalwood) and nineteen (19) grams of essential oil from vetivera zizanioides (vetiver) were combined and added as a fragrance to the palm kernel oil while the palm kernel oil was in its molten state, and thoroughly incorporated with the palm kernel oil.

The palm kernel oil/emollient/fragrance mixture was added to a separatory funnel surrounded by a heating jacket maintained at a temperature of 42° C. The separatory funnel had previously been placed above a cool surface (i.e. granite, marble, steel) and a waxed paper placed between the funnel and the cooled surface. The stopcock of the funnel was adjusted to allow for a flow of the molten mixture to form drops on the waxed paper of a mass approximately one-tenth (0.10) of one gram. These drops formed wax pastilles. The waxed paper sheet was slowly moved from side to side and lengthwise to allow the wax pastilles to form independently and to allow all of the molten material in the funnel to completely drain. This process was for research and standard wax pastille processes would be used for production.

The wax pastilles were allowed to completely harden. Once they had hardened they were packaged in a metal container with a slip lid.

To use, a measured amount of the wax pastilles were placed into the bowl of an aroma lamp used by aroma therapists. The tea-light under the wax pastilles was lit. After a short period, a small amount of the solid oil became molten and the temperature was appropriate for using small amounts for massage, massage therapy, aroma therapy and skin conditioning.

In this case the remaining wax pastilles were already solid and there was no waiting for a return to a solid state for transport.

### EXAMPLE 4

Lavender Scented Candle for Delivery of Massage Oil

50 grams of wax blend

1 ml of lavender essential oil (fragrance)

The wax blend is a blended material of hydrogenated soybean oil and palm kernel oil with a melting point of approximately 49° C. (120° F.). The above ingredients were combined together as in Example 1 and used and stored as described in Example 1.

### EXAMPLE 5

Mango Scented Sweetened Candle for Delivery of Massage Oil

50 grams of wax blend

1 ml of edible mango fragrance oil as a fragrance

2 micrograms of Neotame (an artificial sweetener) as a flavorant

The wax blend is a blended material of hydrogenated soybean oil and palm kernel oil with a melting point of approximately 49° C. (120° F.). The above ingredients were combined together as in Example 1 and used and stored as described in Example 1. 20 micrograms of Alitame or 200 micrograms of Aspartame could have been substituted for the 2 micrograms of Neotame in this and other sweetened examples.

### EXAMPLE 6

Chocolate Scented Sweetened Candle for Delivery of Massage Oil

40 grams of wax blend

10 grams of cocoa butter (acting as a massage oil and as a flavorant and chocolate simulator)

1 ml of edible chocolate fragrance oil as a fragrance

2 micrograms of Neotame (an artificial sweetener) as a flavorant

The wax blend is a blended material of hydrogenated soybean oil and palm kernel oil with a melting point of approximately 49° C. (120° F.). The above ingredients were combined together as in Example 1 and used and stored as described in Example 1.

This disclosure is provided to reveal a preferred embodiment of the invention and a best mode for practicing the invention. Having thus described the invention in this way, it should be apparent that various different modifications can be made to the preferred embodiment without departing from the scope and spirit of this invention disclosure. When structures are identified as a means to perform a function, the identification is intended to include all structures which can perform the function specified.

1- A method for applying a skin preparation, including the steps of:
providing a skin preparation which is in a solid state at room temperature;
melting the skin preparation into a liquid state;
applying the liquid skin preparation to a skin surface; and
rubbing the liquid skin preparation into the skin surface.

2- The method of claim 1 wherein said providing step includes the step of configuring the skin preparation to include at least one oil that is solid at room temperature.

3- The method of claim 2 wherein said configuring step includes the step of selecting the oil from a group of oils including one or more of hydrogenated soybean oil, palm kernel oil, coconut oil and cocoa butter.

4- The method of claim 2 wherein said configuring step includes the step of adding a fragrance to the preparation.

5- The method of claim 2 wherein said configuring step includes the step of adding a flavorant to the preparation.

6- The method of claim 1 wherein said applying step includes the step of pouring the liquid skin preparation onto the skin surface.

7- The method of claim 1 wherein said rubbing step includes the step of massaging the liquid skin preparation into the skin surface.

8- The method of claim 7 wherein said rubbing step includes the step of using hands to massage the liquid skin preparation into the skin surface.

9- The method of claim 1 wherein said melting step includes the steps of providing a wick within the skin preparation, and igniting the wick, such that a flame is produced capable of melting the skin preparation from a solid state into a liquid state.

10- The method of claim 1 wherein said melting step includes the step of applying heat to the skin preparation by radiation heat transfer.

11- The method of claim 1 wherein said melting step includes the step of applying external heat by conduction heat transfer.

12- The method of claim 1 including the further step of placing the skin preparation within a metal container through which heat transfer can readily occur and which can contain the skin preparation when it has melted into a liquid state.

13- A method for storing and using massage oil, including the steps of:
providing a massage oil preparation which is in a solid state in a majority of high temperature atmospheric conditions;
melting the massage oil preparation into a liquid state;
applying the massage oil preparation to a skin surface; and
massaging the massage oil into the skin surface.

14- The method of claim 13 wherein said providing step includes the step of choosing a massage oil preparation having a melting temperature at standard pressure of greater than 100° F.

15- The method of claim 14 wherein said choosing step includes the massage oil preparation having a melting temperature between 80° F. and 130° F.

16- The method of claim 15 wherein said choosing step includes the massage oil preparation having a melting temperature between 110° F. and 125° F.

17- The method of claim 16 wherein said choosing step includes the massage oil preparation having a melting temperature of substantially about 120° F.

18- The method of claim 13 including the further step of configuring the massage oil to include one or more of hydrogenated soybean oil, palm kernel oil, coconut oil and cocoa butter.

19- The method of claim 18 wherein said configuring step includes the further step of adding a fragrance to the massage oil preparation.

20- The method of claim 19 wherein said configuring step includes the further step of adding an emollient to the massage oil preparation.

21- The method of claim 20 wherein said configuring step includes the further step of adding a flavorant to the massage oil preparation.

22- The method of claim 21 wherein said configuring step includes the step of adding cocoa butter to the massage oil preparation, selecting the fragrance to include edible chocolate fragrance oil and selecting the flavorant to include Neotame.

23- A sweetened massage oil composition which is solid at room temperature and a liquid at an elevated skin safe temperature, comprising in combination:
at least one oil having a melting point at standard pressure of between about 80° F. and 130° F.;
a fragrance producing substance; and
a sweetener.

24- The composition of claim 23 wherein said at least one oil includes soy wax.

25- The composition of claim 24 wherein said at least one oil includes a majority soy wax.

26- The composition of claim 25 wherein said composition includes cocoa butter.

27- The composition of claim 26 wherein said composition includes edible chocolate fragrance oil.

28- The composition of claim 27 wherein said composition includes edible chocolate fragrance oil in an amount up to about three milliliters for each approximately forty grams of soy wax, and approximately ten grams of cocoa butter for each approximately forty grams of soy wax.

29- The composition of claim 27 wherein said composition includes Neotame as at least a portion of said sweetener.

30- The composition of claim 29 wherein said composition includes at least about two micrograms of Neotame for each forty grams of soy wax.

31- The composition of claim 25 wherein said oil is at least ninety percent soy wax.

32- The composition of claim 25 wherein said composition includes edible mango fragrance oil.

33- The composition of claim 32 wherein said composition includes about one milliliter of edible mango fragrance oil for each fifty grams of soy wax.

34- The composition of claim 33 wherein said composition includes Neotame as at least a portion of said sweetener.

35- The composition of claim 34 wherein said composition includes at least about two micrograms of Neotame for each fifty grams of soy wax.
